(19) United States
(12) Patent Application Publication
(10) Pub. No.: US 2011/0301627 A1
(43) Pub. Date: Dec. 8, 2011

(54) Device and Method for Conveying a Liquid for Medical Purposes

(76) Inventors: Konrad-Wenzel Winkler, Warin (DE); Andreas Runow, Schwerin (DE)

(21) Appl. No.: 13/201,935

(22) PCT Filed: Feb. 10, 2010
(86) PCT No.: PCT/EP2010/051634

§ 371 Date: Aug. 17, 2011

(30) Foreign Application Priority Data
Feb. 17, 2009 (DE) 10 2009 009 231.5

(51) Int. Cl. A61B 17/3203

(52) U.S. Cl. 606/167

(57) ABSTRACT

The object of the invention is therefore to improve the safety management of a generic device and a corresponding method for conveying a liquid. To this end, it is proposed that the measuring device and the regulating device are constructed in such a way that the safety circuit of the regulating device switches off the pump when the liquid flow falls below a lower limit value Iₘᵢₙ.

The invention is directed to a device for conveying a liquid comprising a driven pump, an input device for a reference value of at least one parameter characterizing the liquid flow, a measuring device for the actual value of the parameter, and a regulating device for determining the control difference and for delivering a control variable for the pump, wherein the regulating device is outfitted with a safety circuit.

The invention is further directed to a method for conveying a liquid for medical purposes in which the actual flow rate of the liquid flow is determined and compared to an entered reference value and is corrected in case of a deviation from the reference value.

Devices and methods of the type mentioned above are used in medical technology.

In medical technology there is a range of applications in which a liquid flow comes into contact with the human body. In this connection, this liquid flow must possess certain parameters and meet conditions to accomplish its purpose on the one hand but, on the other hand, must not endanger or injure the human body. The corresponding device must be outfitted with reliable safety management to avert dangers and injuries.

For example, an electrically driven roller hose pump which conveys human blood in an extracorporeal circulatory system via a blood purification machine is known from DE 25 35 650. In order to ensure a substantially constant pump output in conjunction with certain external conditions, the flow rate of the blood flow is determined and displayed by means of the electric power consumption of the electric motor. Corrections can be made to the pump output of the roller pump by comparison with a predetermined flow rate.

DE 10 2004 028 361 B3 describes a rinsing device for endoscopy for cleaning an operating field in a human body or the endoscope optics located in the region of the operating field. To avoid endangering the patient, for example, due to the rupture of a tube of which a portion is located in the human body, the flow rate of the roller pump is constantly determined by way of the power consumption of the electric motor and is reduced or even interrupted when a critical value is reached by regulating the voltage of the motor driving the roller pump.

Another case of application is shown in DE 203 09 616 U1 which describes a water jet device for severing a biological structure. The construction of this severing device with respect to instrument technology corresponds to the construction of the rinsing device described above, but a piston pump is used instead of a roller pump. In view of the different purpose, the severing device only operates in the higher pressure range. This calls for particular safety. With regard to practical construction, the severing device is then also outfitted with a regulating device which likewise determines the flow rate of the liquid flow via the power consumption of the electric motor driving the piston pump and reduces or even switches off the output of the pump by varying the voltage to the motor.

All of these devices share the disadvantage that it goes unnoticed when the liquid flow falls below a lower limit value. A drop in the liquid flow below the lower limit value may be related, for example, to a drop in output in the pump due to an internal leak, or to air entering the liquid flow, or to an interruption in the water supply. For example, air can enter the liquid flow due to external leaks in the suction area of the device or when the supply reservoir is empty or improperly changed. The operator is generally unaware when the liquid flow decreases impermissibly or when air enters, so it may happen that air, with all of its harmful effects, can subsequently enter the human body, tissue parts are suctioned off dryly because cutting cannot take place due to the absence of liquid, and, on top of this, the patient is deprived of medication contained in the liquid. All of this causes considerable stress on, or injury to, the patient's body.

Therefore, the invention has the object of improving the safety management of a generic device and a corresponding method for conveying a liquid.

This object is met in that the measuring device and the regulating device are constructed in such a way that the safety circuit of the regulating device switches off the pump when the liquid flow falls below a lower limit value Iₘᵢₙ.

The novel device and the novel method overcome the above-mentioned disadvantages of the prior art.

In this respect, it is particularly advantageous that a safeguard it now also provided for a drop in the flow rate below a permissible lower limit value. Contrary to when an upper limit of the output range is impermissibly exceeded, where the pump can generally be controlled so as to be reduced to the permissible output range again, the pump is generally switched off when there is a drop below a lower limit. Simply increasing the pump output would only lead to increased introduction of air. Accordingly, the possibility is afforded to find the causes of the reduction in output and eliminate them without endangering or injuring the patient. This enhances the quality of medical practice and protects the patient. In this connection, the novel device is constructed in a simple manner and is accordingly simple to manufacture. Therefore, production costs remain low. An existing device can also be retrofitted with the novel features, which results in substantial savings. Moreover, the novel device is easy to operate and the novel method is easy to apply.

Advisable embodiments are indicated in the subclaims 2, 3, 4, 6 and 7.

The invention will be described more fully with reference to two embodiment examples.

The drawings show:

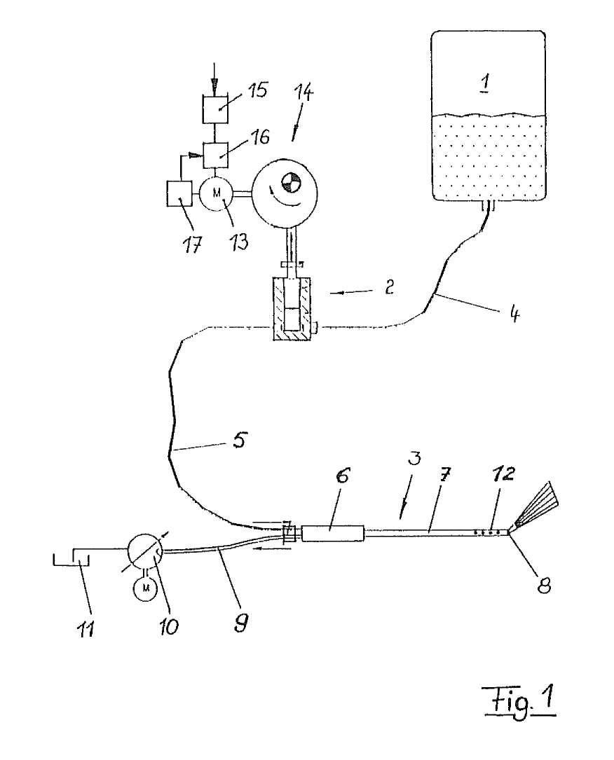

FIG. 1 a device for conveying a liquid flow as severing device in a first embodiment form;

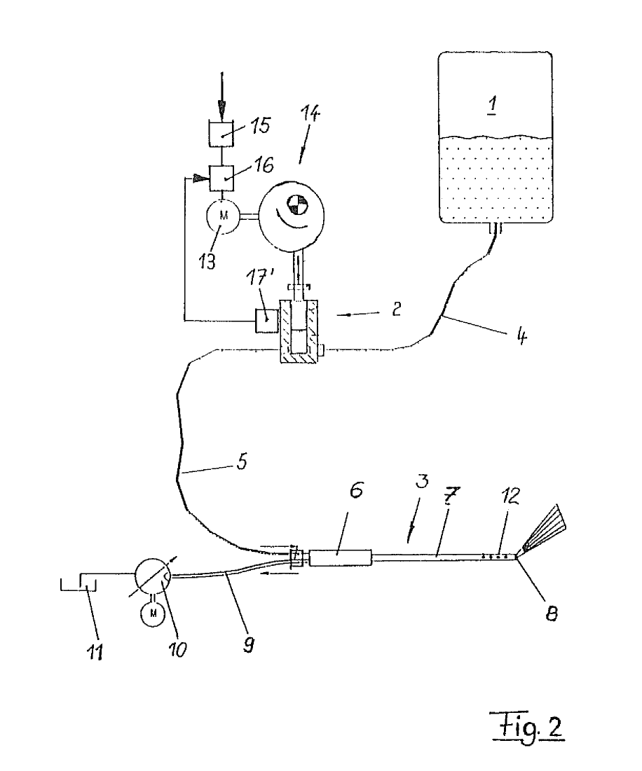

FIG. 2 a device for conveying a liquid flow as severing device in a second embodiment form; and

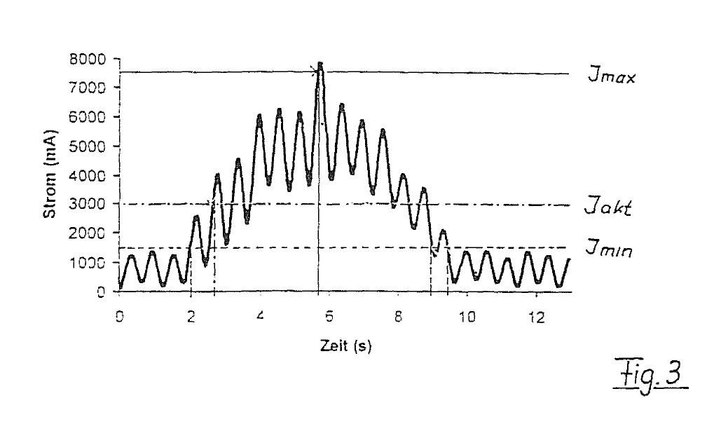

FIG. 3 a load current/time graph of the device for conveying a liquid flow.

According to FIGS. 1 and 2, the water jet device for severing a biological structure comprises a supply reservoir 1 for the severing liquid to be used, a hydraulic pump 2 in the form of a piston-cylinder unit, and an operating handpiece 3. The piston-cylinder unit 2 is connected to the supply reservoir 1 by a suction line 4 on one side and to the operating handpiece 3 by a pressure line 5 on the other side.

In a known manner, the operating handpiece 2 comprises a handpiece 6 and a pressure tube/suction tube 7. The pressure tube/suction tube 7 has an internally located pressure cannula having an outlet nozzle 8 which is arranged at the end and which communicates with the pressure line 5 leading to the pump 2. Further, the pressure tube/suction tube 7 has a suction tube which encloses the pressure cannula while forming an annular channel and which is connected to a collecting tank 11 via a suction extraction line 9 and a driven suction pump 10. In addition, the suction tube further comprises radial suction openings 12 which are arranged so as to be distributed along its circumference for receiving the severed tissue particles and the accumulated severing liquid.

The pump 2 is connected to a preferably electric drive 13 and a gear unit 14.

The drive 13 is outfitted with an input device 15 and with a regulating device 16. The input device 15 is constructed in such a way that a reference value of one or more parameters which characterizes the flow rate of the desired liquid flow can be entered. The regulating device 16 is located between the input device 15 and the drive 13 and is constructed in such a way that the entered reference value as a control variable for activating the drive 13 and an incoming measurement signal can be evaluated and transformed into a control variable for adjusting the drive 13. The required measurement signal for adjusting the drive 13 is generated by an analog or digital measuring device 17.

According to FIG. 1 for the first embodiment form of the device for conveying a liquid, this measuring device 17 is constructed for measuring the electric current strength of the drive 13 of the pump 2 and is accordingly connected on the input side to the drive 13 and on the output side to the regulating device 16.

In continuous operation of the pump 13, the measuring device 17 determines the actual electric current strength of the drive 13 and transmits it to the regulating device 16 which performs a conversion to the rate of flow of the liquid and compares this actual value to the entered reference value of the rate of flow. When an upper limit value of the permissible rate of flow is exceeded, the voltage of the drive 13 is adjusted downward to adjust the actual rate of flow again to the permissible rate of flow. In this regard, instead of adapting the actual value to the reference value, the drive 13 can also be switched off. When the rate of flow falls below a lower limit value, the pump 2 is switched off immediately or after a delay.

According to FIG. 2 for the second embodiment form of the device for conveying a liquid, the measuring device 17′ is designed to measure a hydraulic measurement quantity of the liquid flow. Accordingly, the measuring device 17′ can be a hydraulic flow rate measuring device, for example. But the measuring device can also be an optical detector or ultrasound detector for detecting air bubbles in the liquid which allow inferences to be made concerning the flow rate. In the present embodiment form, this measuring device 17′ is located in the region of the hydraulic chambers of the pump 2. In a corresponding design, the measuring device 17′ can also be arranged in the region of the suction line 4 or pressure line 5.

In continuous operation of the pump 13, the measuring device 17′ determines the actual flow rate and transmits it to the regulating device 16 which compares this actual value to the entered reference value of the flow rate. When an upper limit value of the flow rate is exceeded, the voltage of the drive 13 is adjusted downward, as was already described, or is switched off. When the rate of flow falls below a lower limit value, the pump 2 is switched off immediately or after a delay.

The function of the device and of the corresponding method for conveying a liquid will now be described with reference to FIG. 3 which shows a load current-time graph for an electrically driven pump 2 according to the first embodiment form. The graph also reveals the safety management overall.

When the device and, therefore, the pump 2 are put into operation, the amounts of air found in the system are first pressed out on the pressure side. In so doing, only a negligible pressure builds up in the pressure line so that the pump 2 is loaded only slightly. For this purpose, the pump 2 needs only a relatively small electrical current strength below 1000 mA. After about 2 seconds, the deaeration is terminated and the system is filled with the liquid. This causes a pressure to be built up on the pressure side which results in an increased load on the operation of the pump 2. The power requirement of the electric drive 13 increases and exceeds the lower limit Iₘᵢₙ of the normal output range of the pump 2. In this operating state, a liquid flows at a permissible, predetermined flow rate.

With another rise in electrical current strength, the power consumption curve reaches an activation limit Iₐₖₜ after about 2.5 seconds which is empirically fixed beforehand and which is approximately 3000 mA in the present instance. The first time this activation limit Iₐₖₜ is exceeded, the safety management of the device is activated and ensures that the pump 2 always operates in the normal output range between the lower limit Iₘᵢₙ and an upper limit Iₘₐₓ.

When an upper limit Iₘₐₓ of about 7500 mA is exceeded, which takes place in the present instance after about 6 seconds and which can occur due to an operator error or an unwanted narrowing in cross section or blockage of the pressure line 5, an endangerment of or injury to the patient results. The regulating device 16 detects that the limit is exceeded and switches off the pump 2. The reason for this overload must then be determined and is to be found only in the hydraulic system.

In case of an unwanted reduction in the flow rate, which can also occur due to air pockets in the liquid, a drop in power occurs in the electric drive 13 which allows the load current curve to drop below the lower limit Iₘₘ in the selected example after about 9 seconds. Endangerment of or injury to the patient also occurs when the power range falls below the lower limit Iₘᵢₙ of the normal power range so that voltage to the electric drive 13 is immediately cut off and the conveying of the liquid is halted. Depending on the application, this cutting off can be initiated quickly or with a delay. For example, the device switches quickly the first time there is a drop below the lower limit Iₘᵢₙ. It switches with a delay when the load current remains below the lower limit Iₘᵢₙ over several pumping cycles of the pump 2.

### REFERENCE NUMERALS

1 supply reservoir
2 pump
3 operating handpiece
4 suction line
5 pressure line
6 handpiece
7 pressure tube/suction tube
8 outlet nozzle
9 suction extraction line
10 suction pump
11 collecting tank
12 suction opening
13 drive
14 gear unit
15 input device
16 regulating device
17 measuring device
17′ measuring device

1. Device for conveying a liquid for medical purposes comprising a driven pump, an input device for a reference value of at least one parameter characterizing the liquid flow, a measuring device for the actual value of the parameter, and a regulating device for determining the control difference and for delivering a control variable for the pump, wherein the regulating device is outfitted with a safety circuit, characterized in that the measuring device and the regulating device are constructed in such a way that the safety circuit of the regulating device switches off the pump when the liquid flow falls below a lower limit value Iₘᵢₙ.

2. The device according to claim 1, wherein the regulating device is adjusted in such a way that the safety circuit is first activated when an output value Iₐₖₜ lies above the lower limit value Iₘᵢₙ.

3. The device according to claim 1, wherein the regulating device is outfitted with a delayed-action safety circuit.

4. The device according to claim 1, characterized in that wherein the pump has an electric drive, and the measuring device and the regulating device are designed to evaluate the voltage and current strength of the drive.

5. The method for conveying a liquid for medical purposes in which the actual flow rate of the liquid flow is determined and is compared to an entered reference value and is corrected in case of a deviation from the reference value, wherein the liquid flow is shut off when it falls below a lower limit Iₘᵢₙ of a permissible flow rate range.

6. The method according to claim 5, wherein the flow rate is first switched off when the load current characterizing the liquid flow lies below the lower limit Iₘᵢₙ of the permissible flow rate range for a predetermined period of time.

7. The method according to claim 6, wherein the liquid flow is first switched off when the load current has previously exceeded an output value Iₐₖₜ lying above the lower limit of the permissible flow rate range.
